(19) United States
(12) Patent Application Publication
(10) Pub. No.: US 2013/0310662 A1
(43) Pub. Date: Nov. 21, 2013

(54) SLEEP EVALUATION DEVICE AND SLEEP EVALUATION METHOD

(71) Applicants: Masakazu Tsutsumi, Kyoto-shi (JP); Feilang Tseng, Kyoto-shi (JP); Yoko Kanemitsu, Ibaraki-shi (JP); Yasuko Emori, Kyoto-shi (JP)

(72) Inventors: Masakazu Tsutsumi, Kyoto-shi (JP); Feilang Tseng, Kyoto-shi (JP); Yoko Kanemitsu, Ibaraki-shi (JP); Yasuko Emori, Kyoto-shi (JP)

(73) Assignee: OMRON HEALTHCARE CO., LTD., Muko-shi, Kyoto (JP)

(21) Appl. No.: 13/982,453

(22) PCT Filed: Dec. 20, 2011
(86) PCT No.: PCT/JP2011/079518

§ 371 Date: Jul. 29, 2013

(30) Foreign Application Priority Data
Mar. 11, 2011 (JP) 2011-054173

(51) Int. Cl. A61B 5/00; A61B 5/11; A61B 5/08

(52) U.S. Cl. CPC A61B 5/4809; A61B 5/08; A61B 5/742; A61B 5/11
USPC 600/301; 600/595

(57) ABSTRACT

After a sleep level is measured, a display field that displays current time, a display field that displays sleeping state duration, a display field that displays an image indicating the occurrence of snoring, a display field for distinguishing and displaying a sleeping state and a waking state in a sleeping duration, a display field that displays an image indicating the occurrence of the waking state during the sleeping duration, and a display field that indicates the duration (waking state duration) in which the waking state occurs during the sleeping duration are displayed on a display device.

### TECHNICAL FIELD

The invention relates to sleep evaluation devices and sleep evaluation methods.

### BACKGROUND ART

Heretofore, various techniques relating to devices for measuring sleep have been disclosed.

For example, Patent Literature 1 (JP 2007-319238A) discloses a technique for measuring temporal changes in the depth of sleep and displaying the measurement results with graphs. Also, Patent Literature 2 (JP 2009-22671A) discloses a technique for displaying a result of measuring biological information such as blood pressure for 12 hours and the like in a radar chart form.

As disclosed in Patent Literatures 1 and 2, even in the case where a measurement result is simply displayed continuously, a specialist can understand trends in the continuous measurement result based on his/her specialized knowledge, and also effectively use the measurement result by comparing the measuring result with that from another day.

### CITATION LIST

Patent Literature

Patent Literature 1: JP 2007-319238A

Patent Literature 2: JP 2009-022671A

### SUMMARY OF INVENTION

### Technical Problem

However, it is difficult for a person who does not have specialized knowledge to understand and effectively use trends in continuous measurement results disclosed in Patent Literature 1 or 2 described above.

The present invention has been made in view of the above circumstances, and it is an object of the present invention to display measurement results on a sleep evaluation device in a mode that can be easily understood.

### Solution to Problem

A sleep evaluation device according to the present invention includes body motion detection means for detecting body motion of a person being measured on a bed, first discrimination means for discriminating a sleeping state of the person being measured in a first period, based on a detection result of the body motion detection means, second discrimination means for discriminating whether the person being measured is in the sleeping state or a waking state for each second period that is longer than the first period, based on a discrimination result of the first discrimination means, and display means for displaying a discrimination result of the second discrimination means on a display device, and the display means further displays information relating to the occurrence of nocturnal awakening in the sleeping state on the display device, based on the discrimination result of the second discrimination means.

Preferably, the information relating to the occurrence of nocturnal awakening is at least duration during which the nocturnal awakening occurs in the sleeping state or the number of times that the nocturnal awakening occurs in the sleeping state.

Preferably, the display means displays a plurality of display portions arranged circularly on the display device, and displays the discrimination result of the second discrimination means using the plurality of display portions, while associating each of the display portions to a fixed period.

Preferably, the display means displays the display portions corresponding to a period that is discriminated to be either the sleeping state or the waking state and the display portions corresponding to a period that is discriminated to be the other state, in different display modes from each other.

Preferably, the display means displays in a lighting manner the display portions corresponding to the period that is discriminated to be either the sleeping state or the waking state, and displays in a blinking manner the display portions corresponding to the period that is discriminated to be the other state.

Preferably, the first discrimination means discriminates the sleeping state of the person being measured for each first period, for a specific period that is longer than the first and second periods, and the display means further displays on the display device cumulative duration of either the sleeping state duration or the waking state duration in the discrimination result of the second discrimination means in the specific period.

Preferably, the second discrimination means further discriminates an awakening state of the person being measured, based on the discrimination result of the first discrimination means, and the display means displays the information relating to the occurrence of nocturnal awakening in the sleeping state on the display device, in response to the second discrimination means discriminating the awakening state of the person being measured.

Preferably, the sleep evaluation device further includes input means that receives input of information designating the end of the specific period, and the display means displays the information relating to the occurrence of nocturnal awakening in the sleeping state on the display device, in response to the input means receiving the input.

Preferably, the sleep evaluation device further includes detection means that detects the occurrence of snoring of the person being measured, and the display means further displays on the display device information indicating that the occurrence of snoring is detected in a case where the detection means detects snoring of the person being measured.

Preferably, the display means further displays the discrimination result of the first discrimination means on the display device.

The sleep evaluation method according to the present invention is a sleep evaluation method that is to be executed in a sleep evaluation device including body motion detection means for detecting body motion of a person being measured on a bed, and the sleep evaluation method includes a step of discriminating a sleeping state of the person being measured in a first period, based on a detection result of the body motion detection means, a step of discriminating whether the person being measured is in the sleeping state or a waking state for each second period that is longer than the first period, based on the discrimination result in the first period, a step of displaying the discrimination result in the second period on a display device, and a step of displaying information relating to the occurrence of nocturnal awakening in the sleeping state on the display device, based on the discrimination result in the second period.

### Advantageous Effects of Invention

According to the present invention, whether a person being measured is in a sleeping state or in a waking state is displayed, and furthermore, information relating to the occurrence of nocturnal awakening based on the result of discriminating type of sleep in the sleeping state is displayed.

In other words, it is possible to recognize an overall state relating to whether the person being measured is in the sleeping state or in the waking state in a period during which the person being measured tries to sleep, by displaying whether the person being measured is in the sleeping state or in the waking state.

Also, even in the case where a person who does not have specialized knowledge looks at the display, he/she can recognize trends in sleep because the information relating to the occurrence of nocturnal awakening is displayed.

Furthermore, it is possible to display whether the person being measured is in the sleeping state or in the waking state as well as the information relating to the occurrence of nocturnal awakening in a reduced display area because whether the person being measured is in the sleeping state or in the waking state is displayed for each fixed time period.

### BRIEF DESCRIPTION OF DRAWINGS

FIG. 1 is a diagram showing a specific example of the external appearance of a sleep level evaluation device (hereinafter, abbreviated to “evaluation device”) serving as a sleep evaluation device according to an embodiment of the present invention.

FIG. 2 is a schematic view representing a lateral face of the evaluation device.

FIG. 3 is the schematic view of the external appearance of the evaluation device seen from diagonally above.

FIG. 4 is a block diagram showing a specific example of the hardware configuration of the evaluation device.

FIG. 5 is a diagram illustrating an exemplary usage of the evaluation device.

FIG. 6 is a block diagram showing a specific example of the functional configuration for discriminating sleep level in the evaluation device.

FIG. 7 is a diagram showing a specific example of a sensor signal output from a body motion sensor that is a Doppler sensor.

FIG. 8A is a diagram showing a specific example of a respiratory waveform separated from the waveform represented in FIG. 7, and FIG. 8B is a diagram showing a specific example of a body motion waveform separated from the waveform represented in FIG. 7.

FIG. 9A is a diagram showing a specific example of discrimination results of a discrimination unit shown in FIG. 6, and FIG. 9B is a diagram showing a specific example of correction of discrimination results shown in FIG. 9A. FIG. 9C is a diagram showing a specific example of sleep level discrimination results for each fixed period.

FIG. 10 is a block diagram showing a specific example of the functional configuration for detecting the occurrence of snoring in the evaluation device.

FIG. 11 is a diagram showing an example of the display modes of a display unit of the evaluation device.

FIG. 12 is a diagram showing an example of the display modes of the display unit of the evaluation device.

FIG. 13 is a diagram showing specific examples of typical waveforms of a sensor signal that correspond to a sleep level.

FIG. 14 is a diagram showing specific examples of typical waveforms of a sensor signal that correspond to a sleep level.

FIG. 15 is a diagram showing specific examples of typical waveforms of a sensor signal that correspond to a sleep level.

FIG. 16 is a diagram showing specific examples of typical waveforms of a sensor signal that correspond to a sleep level.

FIG. 17 is a diagram showing specific examples of typical waveforms of a sensor signal that correspond to a sleep level.

FIG. 18 is a diagram showing specific examples of typical waveforms of a sensor signal that correspond to a sleep level.

FIG. 19 is a diagram showing specific examples of typical waveforms of a sensor signal that correspond to a sleep level.

FIG. 20 is a diagram showing a result of analyzing the normal sound of snoring.

FIG. 21 is a flowchart of processing for discriminating a sleeping state (sleep discrimination processing) of a person being measured in the evaluation device.

FIG. 22 is a diagram showing an example of the display modes of the display unit of the evaluation device.

FIG. 23 is a diagram showing an example of the display modes of the display unit of the evaluation device.

FIG. 24 is a diagram showing an example of the display modes of the display unit of the evaluation device.

FIG. 25 is a diagram showing an example of the display modes of the display unit of the evaluation device.

FIG. 26 is a diagram showing an example of the display modes of the display unit of the evaluation device.

FIG. 27 is a diagram showing an example of the display modes of the display unit of the evaluation device.

FIG. 28 is a diagram showing an example of the display modes of the display unit of the evaluation device.

### DESCRIPTION OF EMBODIMENTS

Embodiments of the present invention will be described hereinafter, with reference to the drawings. In the following description, the same reference signs are given to the same components and constituent elements. The names and functions thereof are also the same.

External Appearance

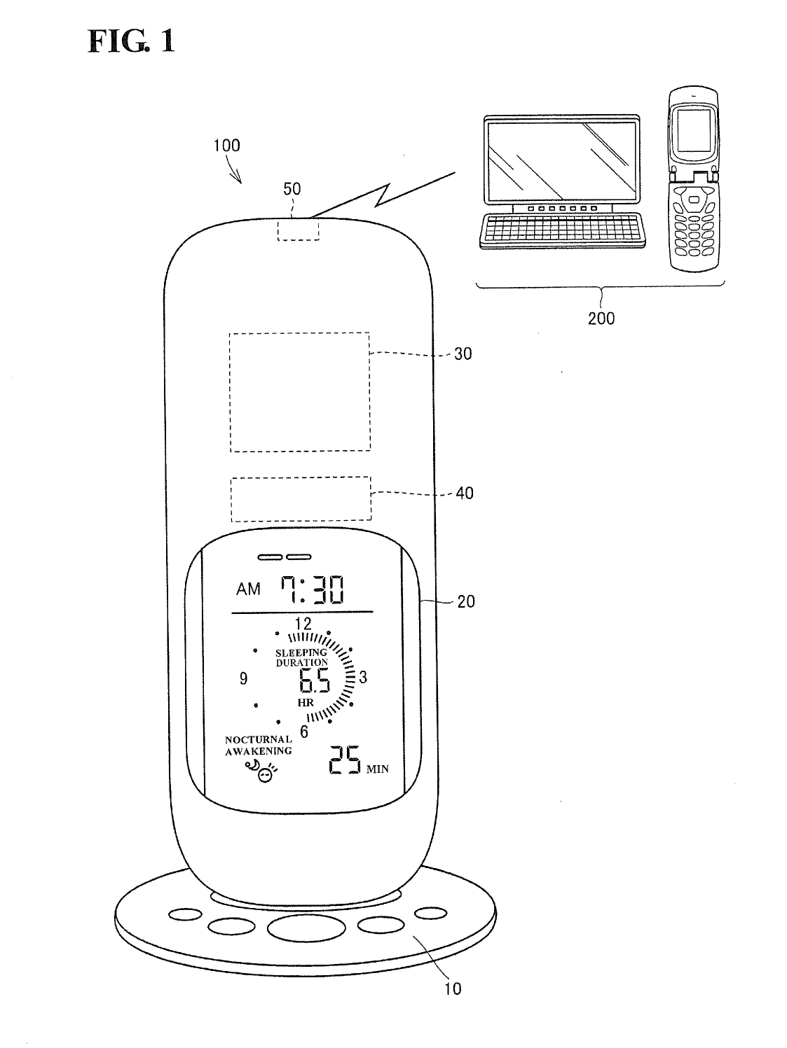

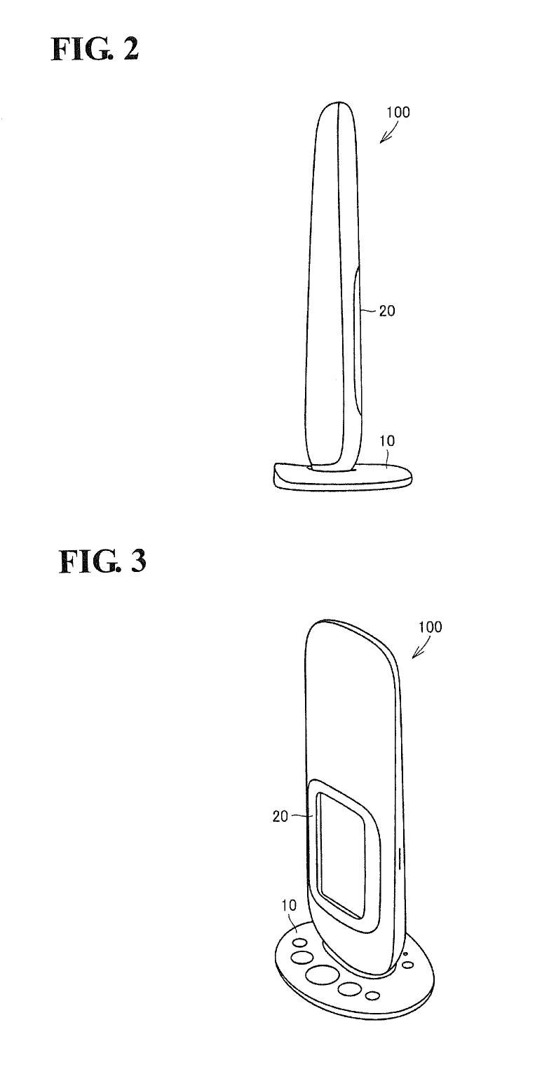

FIG. 1 is a diagram showing a specific example of the external appearance of a sleep level evaluation device (hereinafter, abbreviated to “evaluation device”) 100 according to the present embodiment. Also, FIG. 2 is a schematic view representing a lateral face of the evaluation device 100, and FIG. 3 is a schematic view of the external appearance seen from diagonally above.

Referring to FIGS. 1 to 3, an evaluation device 100 has, as one example, an external appearance in which a casing that is a rectangular parallelepiped or elongated in shape with rounded corners is placed on a base.

Referring to FIG. 1, an operation button group 10 is disposed on the surface of the base, and a display unit 20 is disposed on the surface of the casing that is placed on the base. Also, a sensor 30 and a control unit 40 are incorporated into the casing.

In the subsequent description, the surface of the casing on which the display unit 20 is provided will be called the front face of the evaluation device 100.

The evaluation device 100 has a communication unit 50 for performing wireless or wired communication. The communication unit 50 is, as one example, provided at the opposite end of the casing to the base. The evaluation device 100 is connected to a display device 200 such as a personal computer (hereinafter, PC) or a mobile phone, using the communication unit 50, and outputs display data to the display device 200.

Hardware Configuration

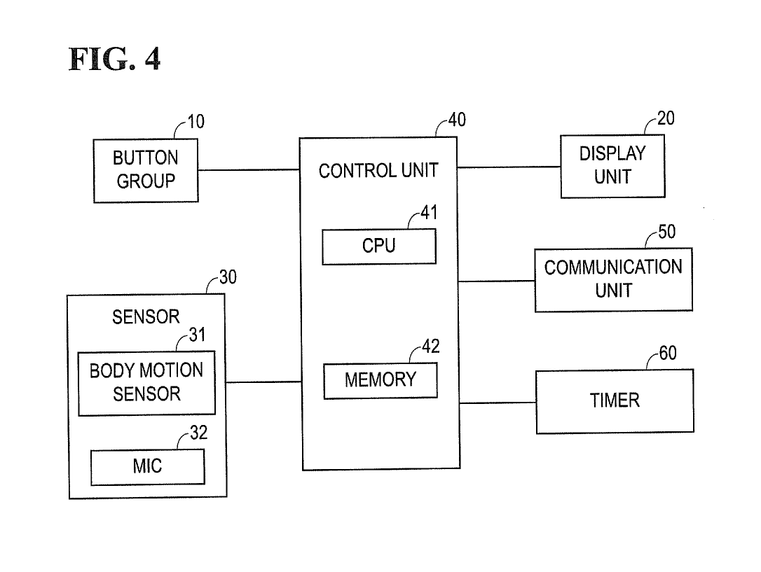

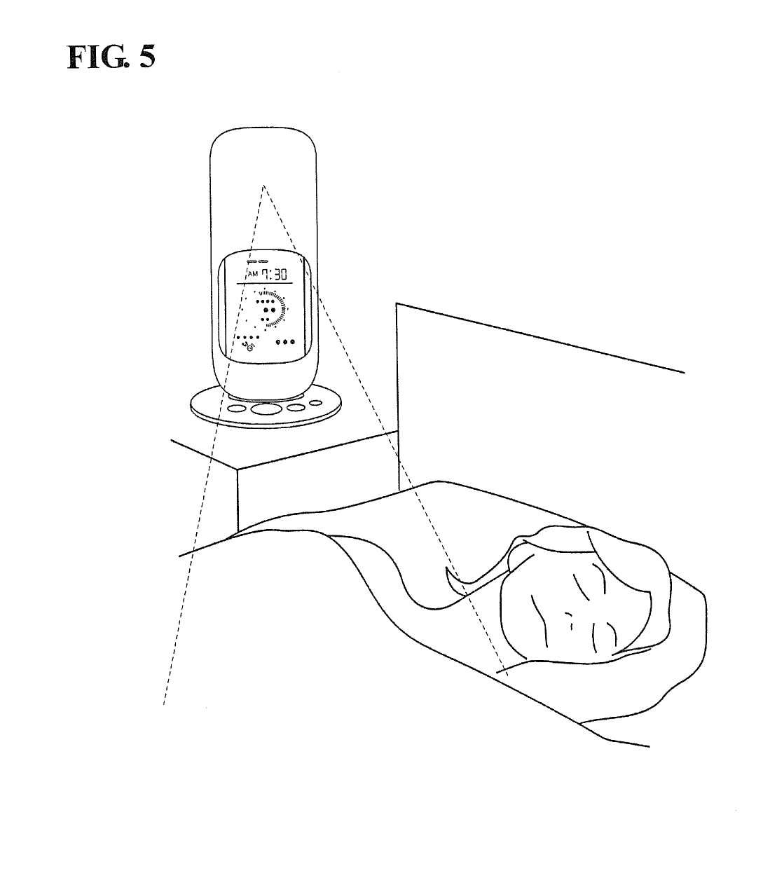

FIG. 4 is a block diagram showing a specific example of the hardware configuration of the evaluation device 100.

Referring to FIG. 4, the button group 10, the sensor 30, the display unit 20 and the communication unit 50 are all connected to the control unit 40.

The button group 10 outputs an operation signal to the control unit 40 as a result of being operated by a person being measured.

The sensor 30 includes a body motion sensor 31 and a microphone (hereinafter, abbreviated to “mic”) 32 that is an example of a sound sensor, and the body motion sensor and the mic respectively output a sensor signal to the control unit 40. A Doppler sensor is preferably used as the body motion sensor 31. In the subsequent description, the body motion sensor 31 is assumed to be a Doppler sensor. Alternatively, an ultrasonic sensor or an infrared sensor may be used.

The body motion sensor 31, which is a Doppler sensor, has an output unit for outputting radio waves for use in measurement and a receiving unit. The receiving unit receives radio waves reflected from the surface of a measurement body among radio waves output from the output unit, and outputs a sensor signal that depends on the change in frequency from the output radio waves.

Note that a camera may be provided instead of the body motion sensor 31 as the mechanism for detecting body motion, and body motion may be detected by performing image analysis in the control unit 40.

The control unit 40 includes a CPU 41 for performing overall control, and a memory 42 for storing programs that are executed by the CPU 41, and the like.

The control unit 40 computes a sleep level discussed later and generates display data for displaying the sleep level, by the CPU 41 executing a program for performing display stored in the memory 42, and executing an operation using an input operation signal and sensor signal. Also, the control unit 40 executes various processes such as computations of sleep latent duration, which will be described later, and the like.

The control unit 40 executes display control for performing screen display on the display unit 20 based on the display data. Furthermore, communication control for transmitting display data from the communication unit 50 to the display device 200 is executed.

The communication unit 50 may communicate directly with the display device 200, by wireless communication such as infrared communication or communication utilizing Bluetooth (registered trademark), for example, or may have an Internet connection function and communicate with the display device 200 via the Internet.

Furthermore, the communication unit 50 may have a wireless LAN (Local Area Network) server function, and transmit display data discussed below that is expressed in a markup language such as HTML (Hypertext Markup Language), for example, to the display device 200 accessed by a wireless LAN connection.

Also, the evaluation device 100 is provided with a timer 60. The timer 60 is connected to the control unit 40. The CPU 41 acquires time information from the timer 60, specifies time such as go-to-sleep time, which will be described later, or the like, and stores the time in the memory 42.

Exemplary Usage

FIG. 5 is a diagram illustrating an exemplary usage of the evaluation device 100.

Referring to FIG. 5, the evaluation device 100 is installed in proximity to the person being measured who is asleep (e.g., bedside) as an example. To perform the measurement operation in this state, radio waves are output from the body motion sensor 31 which is a Doppler sensor.

The radio waves output from the body motion sensor 31 reach mainly the vicinity of the chest and shoulders of the person who is sleeping, and the change in frequency of the waves reflected therefrom is output to the control unit 40 as a sensor signal. The control unit 40 detects body motion, such as chest movement of the person who is sleeping or the person rolling over in his or her sleep, based on the change in frequency, and discriminates the sleep level based on those detection results.

Also, as a result that, the evaluation device 100 is installed in proximity to the person being measured, sound in proximity to the person being measured is detected by a mic 32 that serves as a sound sensor. A sound signal is output to the control unit 40 as a sensor signal. This sensor signal is also referred to as “sound signal” in the description hereinafter.

The control unit 40 detects body motion, such as chest movement of the person who is sleeping or the person rolling over in his or her sleep, based on the change in frequency expressed by a breathing and body motion signal, and discriminates the sleeping state based on those detection results. Also, the control unit 40 detects “snoring” of the person being measured from sound signals, and discriminates the snoring state based on those detection results.

Functional Configuration for Discriminating between Sleeping State and Waking State (Discriminating Sleep Level)

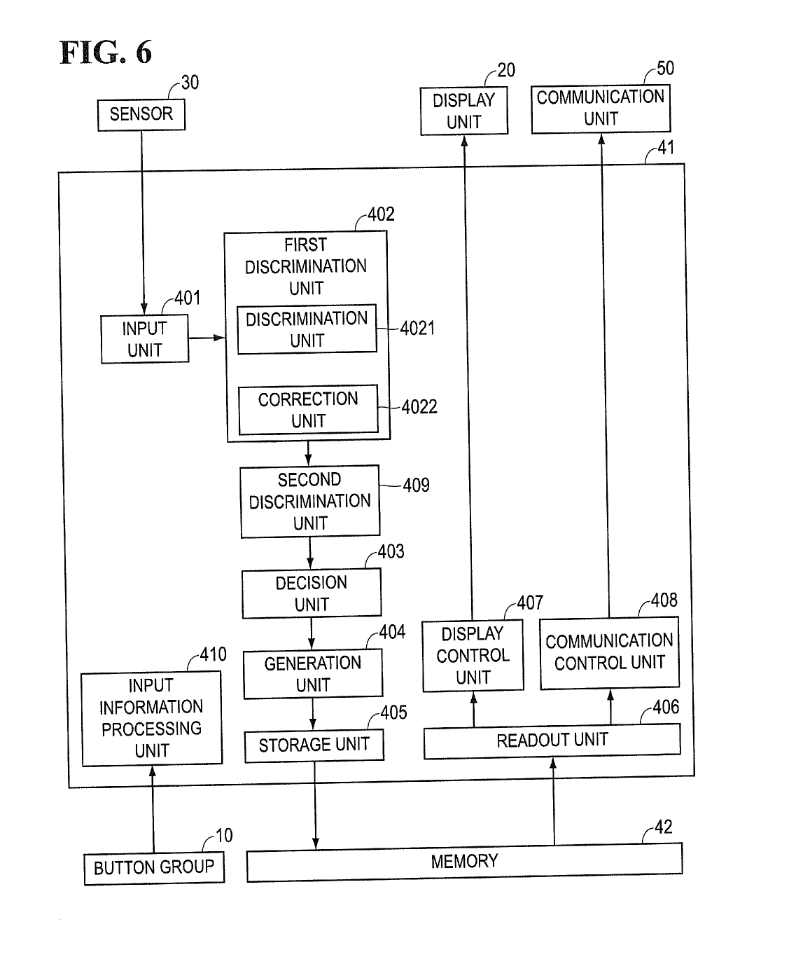

FIG. 6 is a block diagram showing a specific example of the functional configuration for discriminating the sleep level in the evaluation device 100, which is an exemplary functional configuration for discriminating between the sleeping state and the waking state. The functions represented in FIG. 6 are mainly formed on the CPU 41 by the CPU 41 executing programs stored in the memory 42, but at least some of the functions may be formed by a hardware configuration such as electrical circuitry.

Referring to FIG. 6, the evaluation device 100 includes an input unit 401 for receiving input of the sensor signal output from the sensor 30, a first discrimination unit 402 for discriminating the sleeping state of a unit period based on the sensor signal, a second discrimination unit 409 for discriminating a level of the sleeping state in a fixed period consisting of a predetermined number of continuous unit periods, based on a discrimination result for each unit period, a decision unit 403 for deciding a display mode of the fixed period based on the level of the sleeping state, a generation unit 404 for generating display data for displaying the sleep level based on the decided display mode, a storage unit 405 for executing processing for storing display data in the memory 42, a readout unit 406 for reading out display data from the memory 42, a display control unit 407 for executing processing for displaying read display data on the display unit 20, and a communication control unit 408 for executing processing for transmitting display data to the display device 200 using the communication unit 50.

Also, the evaluation device 100 includes an input information processing unit 410 for processing information input from various buttons included in the button group 10.

In the example of FIG. 6, the input unit 401 receives the sensor signal output directly from the sensor 30, but a configuration may be adopted in which the sensor signal is temporarily stored to a predetermined area of the memory 42, and is read out from there by the input unit 401 when performing a display operation.

Sleep Level Discrimination Method

Here, the sleep level discrimination method of the second discrimination unit 409 will be described.

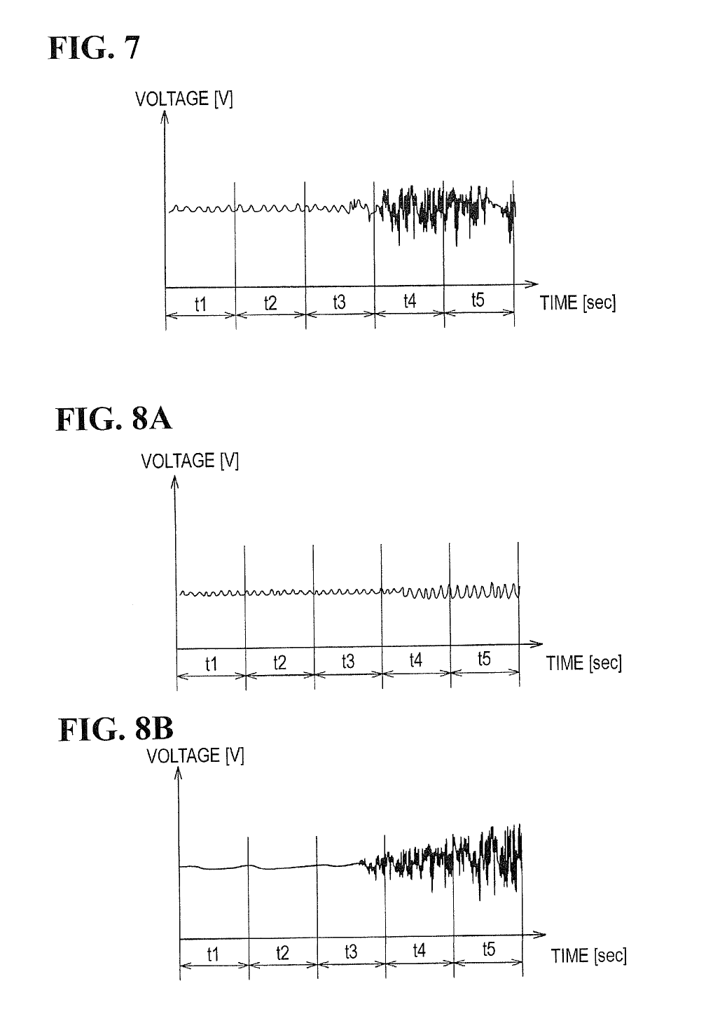

FIG. 7 is a diagram showing a specific example of the sensor signal output from the body motion sensor 31 which is a Doppler sensor. FIG. 7 represents the temporal change in voltage that is related to the phase change between the reflected wave from the body motion sensor 31 and the reflected wave from the surface of the person being measured.

Referring to FIG. 7, the waveform represented by the sensor signal is a composite wave that includes a waveform representing the body motion (chest movement) of the person being measured that is associated with breathing (hereinafter also called a respiratory waveform) and a waveform representing body motion other than breathing such as the person rolling over in his or her sleep or the like (hereinafter also called a body motion waveform).

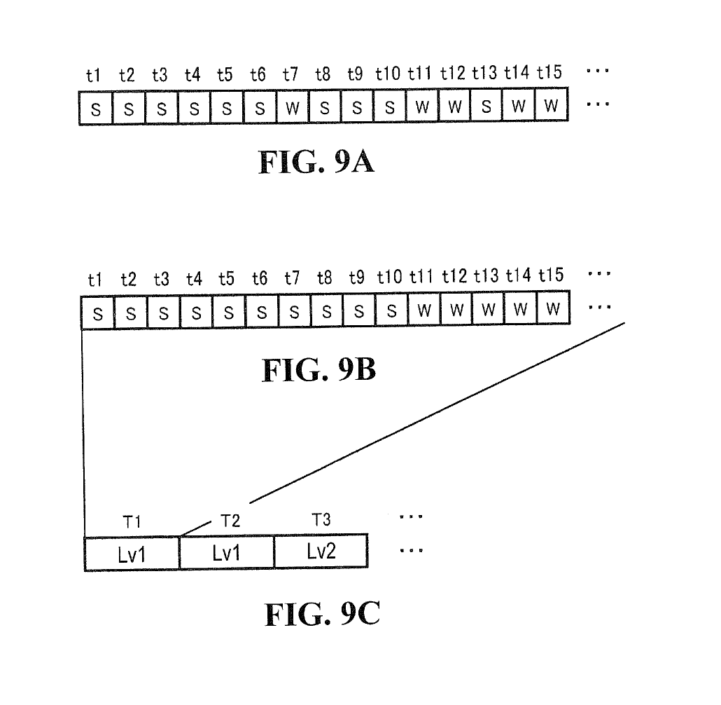

FIGS. 8A-8B and FIGS. 9A-9C are diagrams showing specific examples of a respiratory waveform and a body motion waveform separated from the waveform represented in FIG. 7.

The respiratory waveform of a person who is in a stable sleeping state has periodicity. Accordingly, in the case where the periodicity of the respiratory waveform is within a predetermined range, that is, when variation in the cycle of the respiratory waveform is within a predetermined range, the person can generally be said to be in a stable sleeping state.

Also, when a person is in a stable sleeping state, there is unlikely to be any body motion other than breathing such as rolling over in his or her sleep. Accordingly, a person can generally be said to be in a stable sleeping state when the amplitude of the body motion waveform is within a predetermined range, and can be said to not be in a stable sleeping state in the case where the amplitude is not within the predetermined range, since there is body motion.

Accordingly, it can be discriminated whether or not the person being measured is in a stable sleeping state with regard to a given period, based on the periodicity of the respiratory waveform or the magnitude of body motion other than breathing in that period. Note that although the sleeping state is discriminated using both the respiratory waveform and the body motion waveform in this example, it is possible to use only one of the waveforms.

As shown in FIG. 6, the first discrimination unit 402 includes a discrimination unit 4021 and a correction unit 4022.

The discrimination unit 4021 separates the waveform that is based on the input sensor signal shown in FIG. 7 into the respiratory waveform and the body motion waveform shown in FIGS. 8A-8B and FIGS. 9A-9C. The discrimination unit 4021 then discriminates whether the person being measured is in a stable sleeping state, every prescribed unit period (periods t1, t2, t3, t4, t5 in FIG. 7), based on the respective waveforms. The unit period here is around 30 seconds to 1 minute, for example. That is, if the variation in the cycle in unit period t1 of the respiratory waveform is less than a preset threshold, it is judged that periodicity is evident in the respiratory waveform in unit period t1. Also, it is judged whether the amplitude in unit period t1 of the body motion waveform is greater than or less than a preset threshold.

The discrimination unit 4021 then discriminates that the sleeping state of the person being measured in unit period t1 is a sleeping state (S), in the case where the respiratory waveform has periodicity in unit period t1, and the amplitude of the body motion waveform is less than the threshold. On the other hand, the discrimination unit 4021 discriminates that the sleeping state of the person being measured in unit period t1 is a waking state (W), in the case where the respiratory waveform does not have periodicity in unit period t1, and the amplitude of the body motion waveform is greater than the threshold. Note that the discrimination unit 4021 may be configured to discriminate that the person being measured is in a waking state if only one of these conditions is satisfied, or in other words, if only the respiratory waveform has periodicity in unit period t1 or the amplitude of the body motion waveform is less than the threshold.

Also, the discrimination unit 4021 may discriminate whether or not the person being measured is present within range of radio waves output from the body motion sensor 31.

Such a discrimination may be performed as follows. For example, the waveform that is based on the sensor signal is separated into the respiratory waveform and the body motion waveform as described above, and then in a case where the amplitude of either the respiratory waveform and the body motion waveform continues to be less than a specific value for a specific time period (30 seconds, for example), the discrimination unit 4021 determines that the person being measured is not present in the above-described range. In another case, the discrimination unit 4021 then determines that the person being measured is present in the above-described range. Note that the discrimination unit 4021 discriminates the state of the person's presence or absence as a state (E) if the person being measured is present, or a state (N) if the person being measured is not present.

FIG. 9A is a diagram showing a specific example of discrimination results of the discrimination unit 4021. As shown in FIG. 9A, the discrimination unit 4021 discriminates whether the person being measured is in a stable sleeping state or a waking state, every unit period of the waveform that is based on the input sensor signal.

However, there may also be unit periods where body motion occurs in a sleeping state or where there is no body motion and breathing is regular in a waking state. Also, there may be cases where a reflected wave from a moving object other than the person being measured is received, resulting in noise occurring in the body motion waveform. In view of this, preferably the correction unit 4022 corrects the discrimination result of such unit periods, according to the discrimination results of adjacent unit periods.

As one example, FIG. 9B shows a specific example of correction of discrimination results shown in FIG. 9A. Referring to FIG. 9A and FIG. 9B, in the case where the number of continuous unit periods having the same discrimination result is less than or equal to a predetermined number, and the number of unit periods continuous therebefore and thereafter having the opposite discrimination result is greater than or equal to a predetermined number, the correction unit 4022 corrects the discrimination result of those continuous unit periods having the same discrimination result to the opposite discrimination result.

Specifically, although the discrimination unit 4021 discriminates that unit period t7 in FIG. 9A is the waking state (W), there are no unit periods discriminated to be the waking state (W) that are continuous with unit period t7 (i.e., number of continuous unit periods is 1), and there is a certain number of unit periods continuous before and after unit period t7 that are discriminated to be the sleeping state (S). The unit period t1 3 is also in a similar state where the discrimination result is opposite.

If it is assumed that the threshold (first threshold) for the continuous number of discrimination results of the targeted unit period is 2, and the threshold (second threshold) for the continuous number of discrimination results of unit periods before and after the targeted unit period is 2, these conditions are satisfied for unit period t7 in that the one continuous unit period discriminated to be the waking state (W) is less than the first threshold, and the three continuous unit periods before and after unit period t7 that have the opposite discrimination result are greater in number than the second threshold. Accordingly, the correction unit 4022 corrects the discrimination result of unit period t7 to the sleeping state (S) which is the opposite discrimination result.

Similarly, the correction unit 4022 also corrects the discrimination result of unit period t13 to the waking state (W) which is the opposite discrimination result.

Next, the second discrimination unit 409 discriminates the sleep level for a fixed period consisting of continuous unit periods, based on the discrimination result of each unit period. The unit period here is around 5 minutes to 10 minutes, for example.

Here, the sleep levels denote levels of the sleeping depth defined in terms of the regularity of breathing and the existence and continuity of body motion. Specific examples include:

Level 1: Sleeping state with no body motion and regular breathing;

Level 2: Sleeping state with one-off body motion;

Level 3: Sleeping state with continuous body motion;

Level 4: Waking state with continuous body motion that is ongoing; and

Level 5: Full waking state.

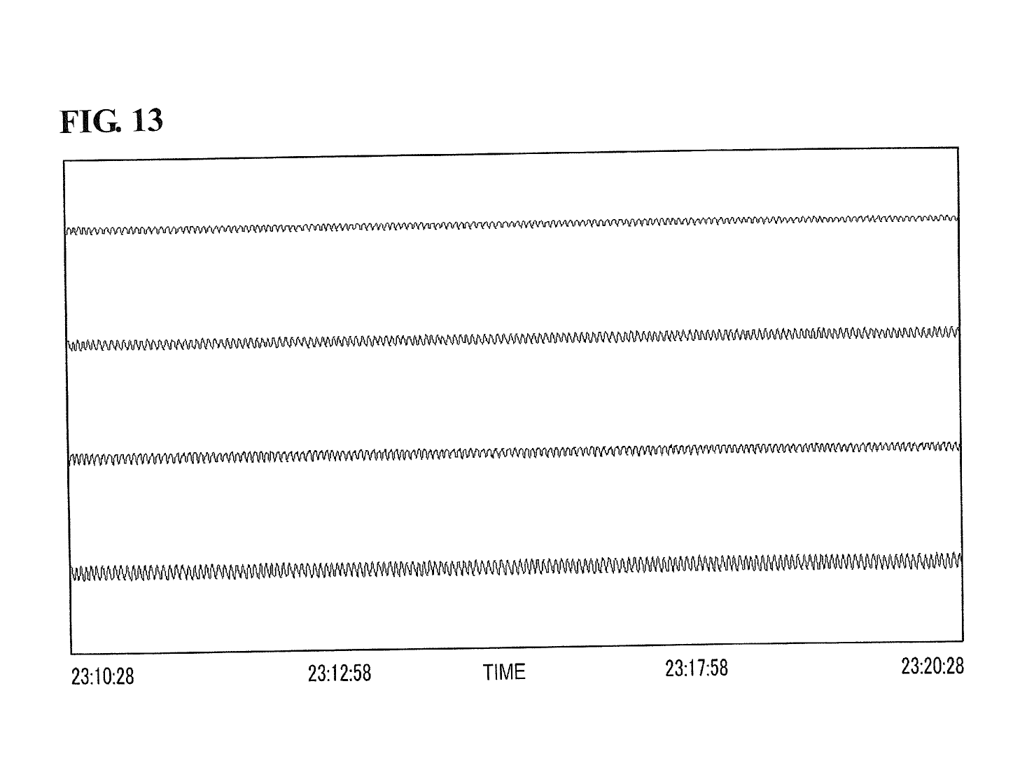

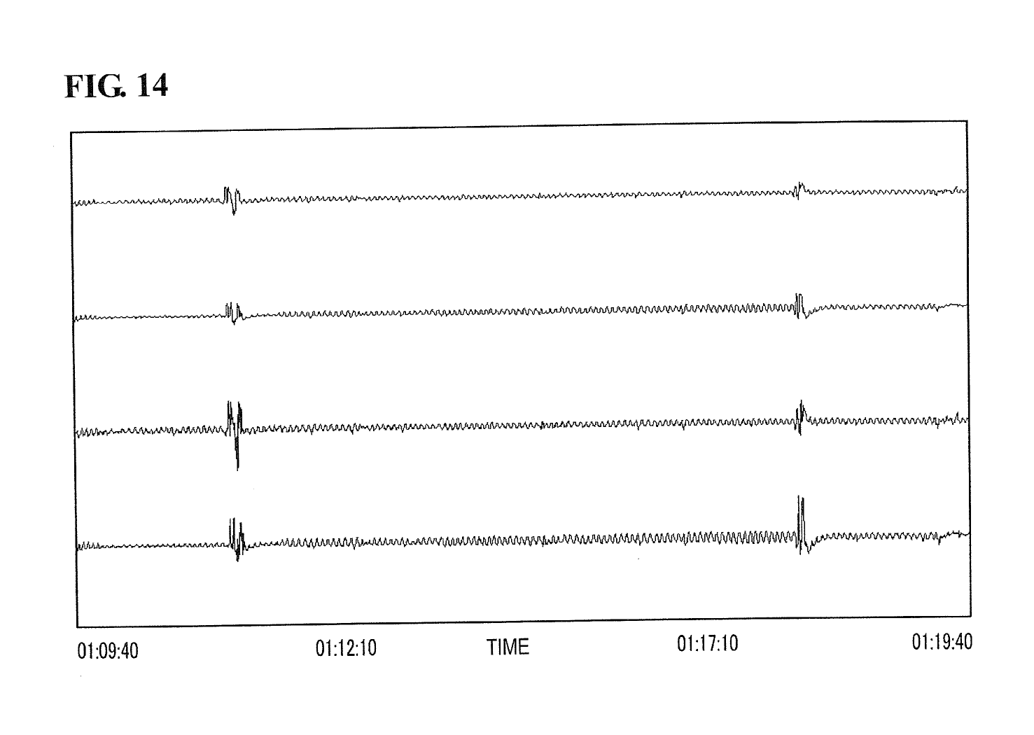

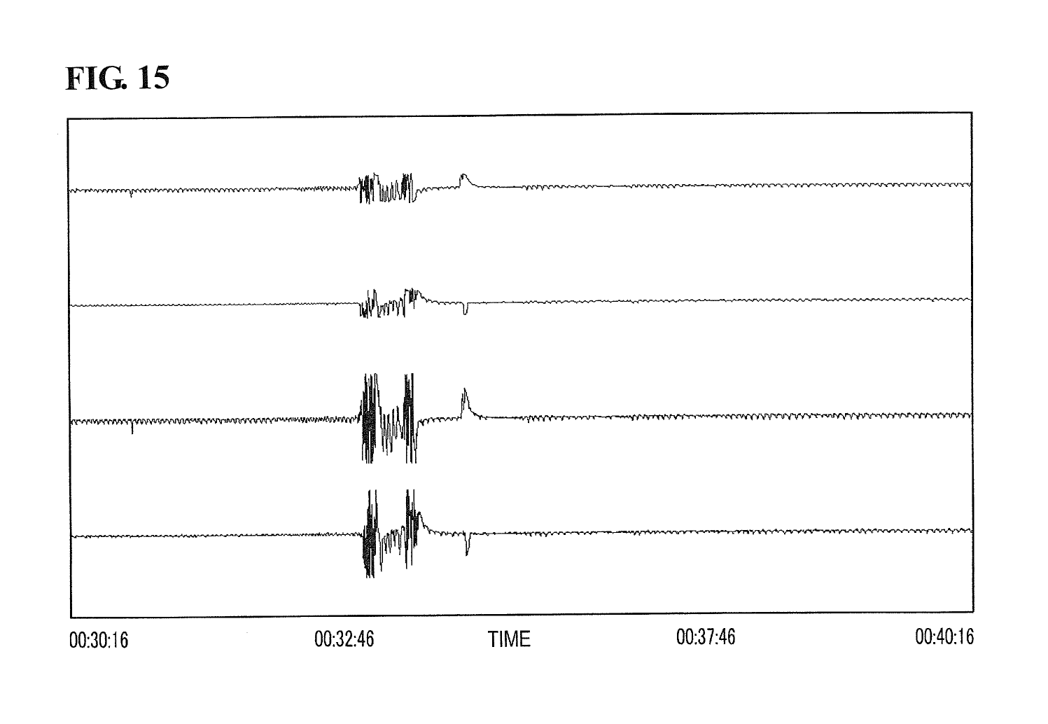

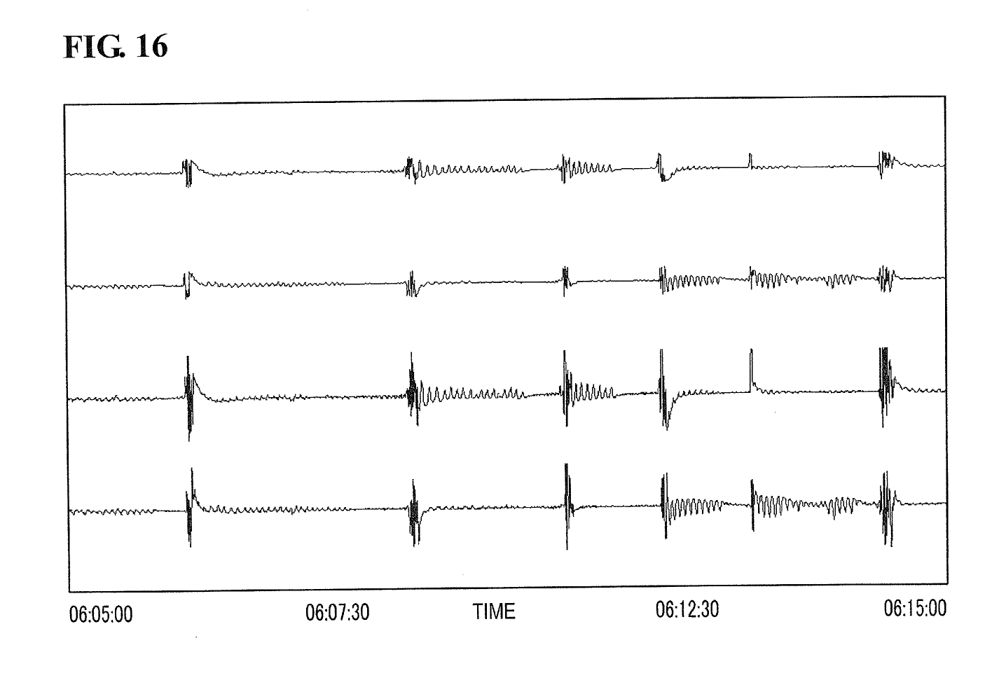

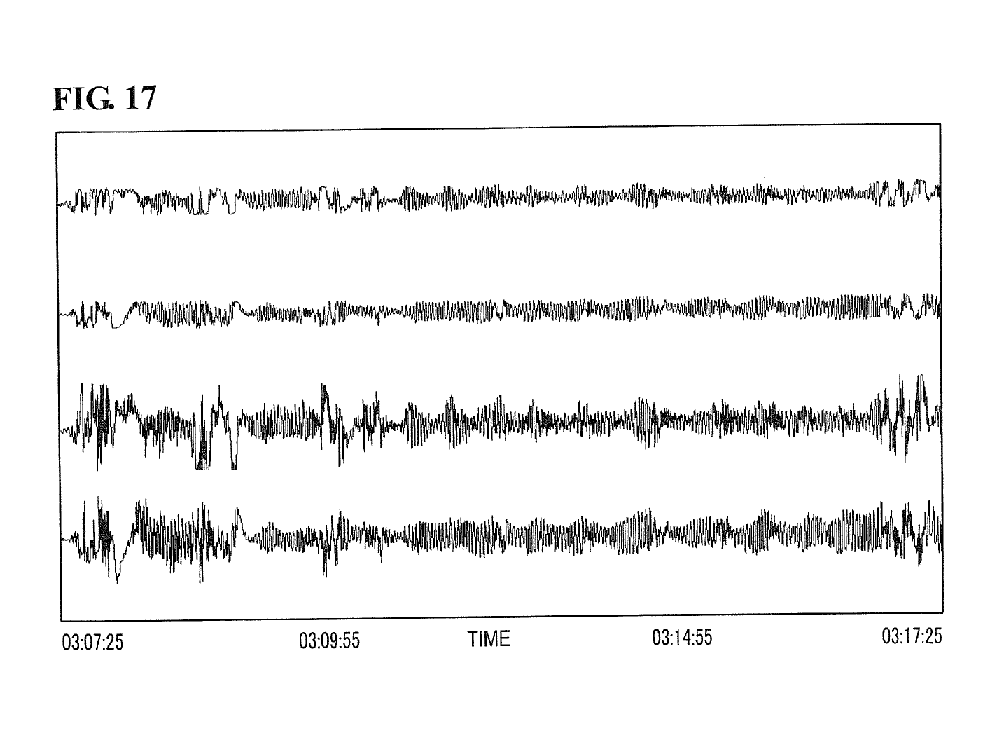

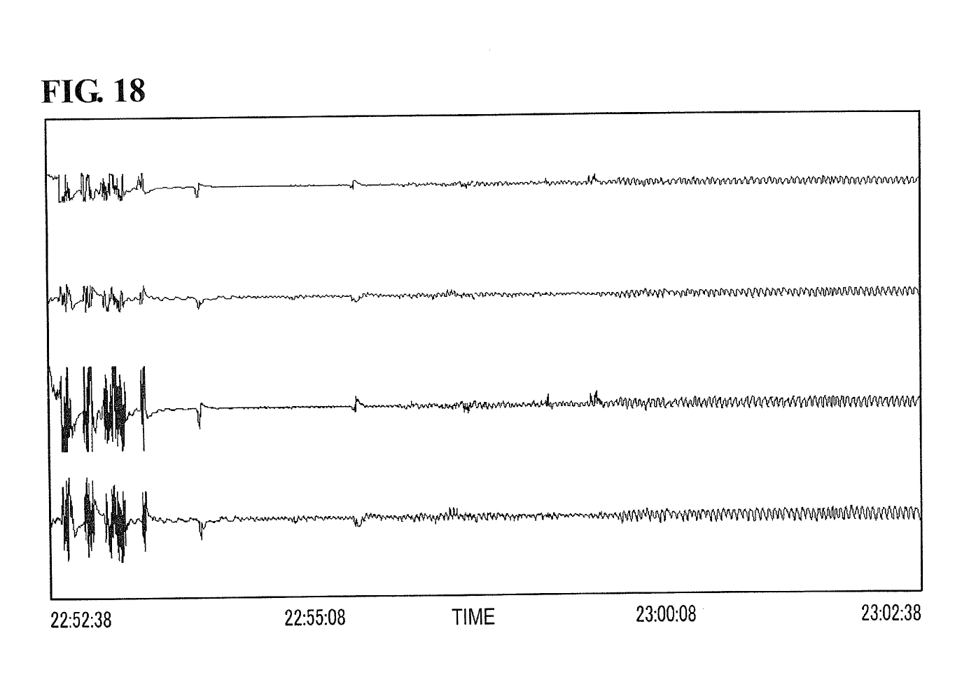

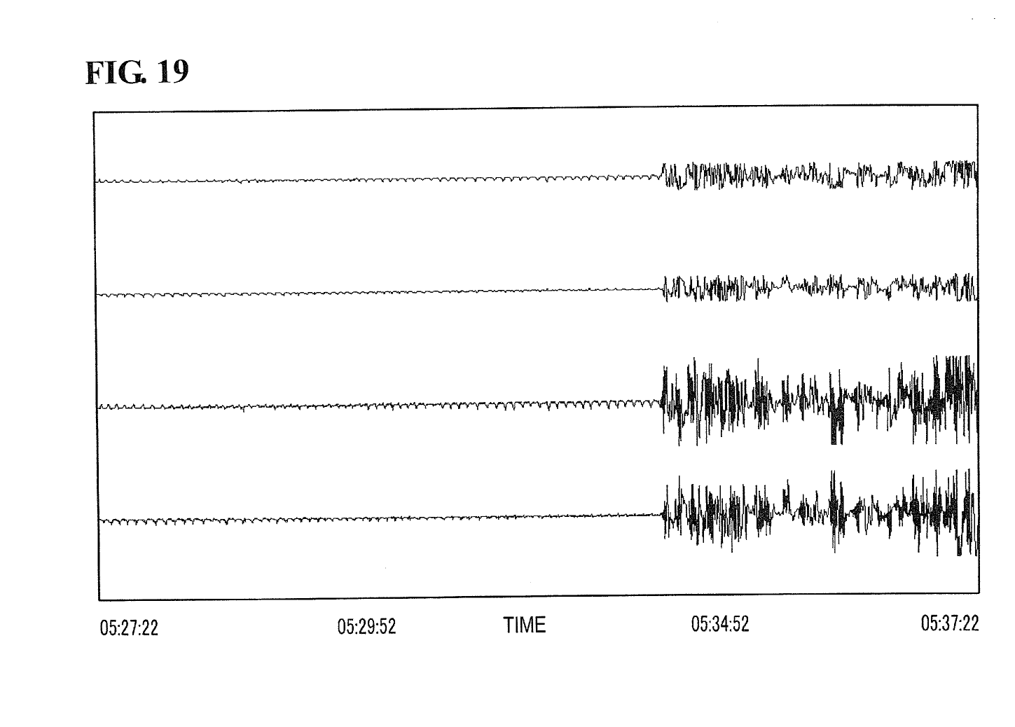

The second discrimination unit 409 stores, as a discrimination value for each level, a continuous number and a ratio of discrimination results of the unit periods constituting a fixed period. As an example, FIG. 13 represents specific examples of typical waveforms of the sensor signal in the case of the above level 1, FIG. 14 represents specific examples of typical waveforms of the sensor signal in the case of the above level 2, FIG. 15 and FIG. 16 represent specific examples of typical waveforms of the sensor signal in the case of the above level 3, FIG. 17 represents specific examples of typical waveforms of the sensor signal in the case of the above level 4, FIG. 18 represents typical waveforms of the sensor signal in the case of the above level 5, particularly waveforms of the sensor signal when the person being measured is going to sleep, and FIG. 19 represents typical waveforms of the sensor signal in the case of the above level 5, particularly waveforms of the sensor signal when the person being measured is waking up. The second discrimination unit 409 prestores, as a discrimination value for each level, a continuous number and a ratio of discrimination results represented in the waveforms of these sensor signals. FIG. 9C is a diagram representing a specific example of the sleep level discrimination result for each fixed period. That is, referring to FIG. 9B and FIG. 9C, the second discrimination unit 409 discriminates the sleep level for each fixed period, by comparing the continuous number of the discrimination result with the stored discrimination value, and comparing the ratio of the discrimination results with the discrimination value, for the continuous unit periods constituting the fixed period.

Functional Configuration for Detecting Snoring

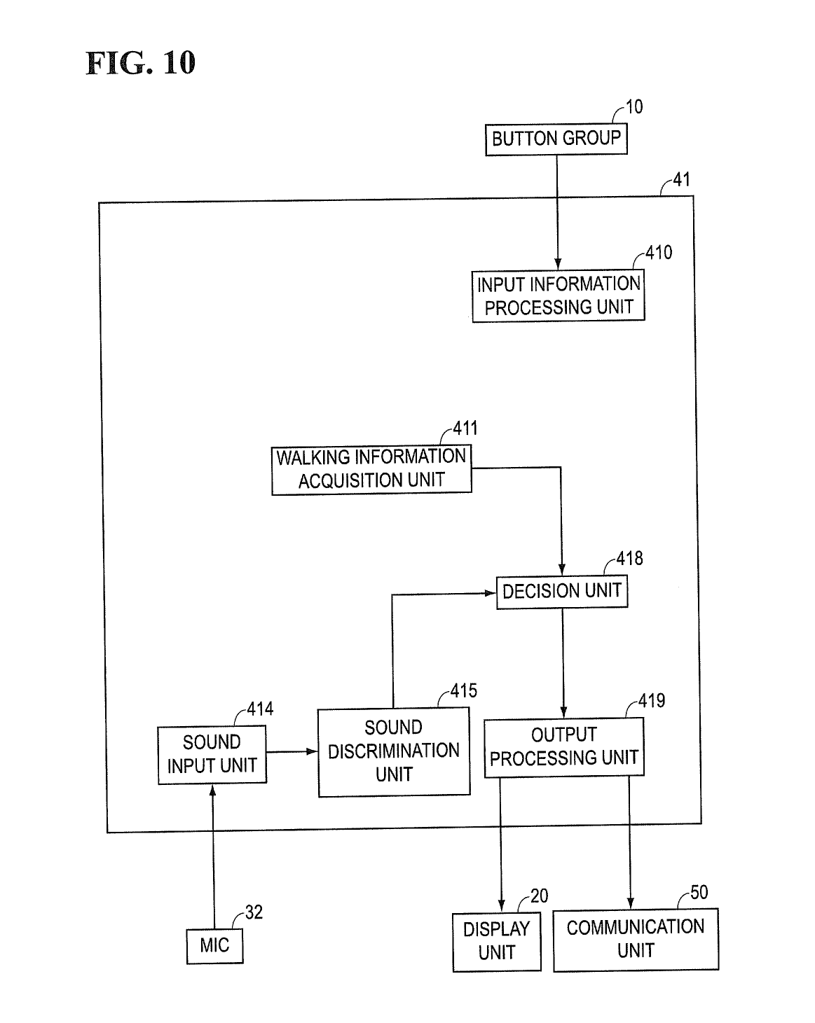

FIG. 10 is a block diagram showing a specific example of the functional configuration for detecting the occurrence of snoring in the evaluation device 100. The functions represented in FIG. 10 are formed on the CPU 41 mainly by the CPU 41 executing programs stored in the memory 42, but at least some of the functions may be formed by a hardware configuration such as electrical circuitry.

Referring to FIG. 10, the evaluation device 100 includes a waking information acquisition unit 411 that acquires a sleep level (level 1 to level 5) discriminated as above for the person being measured at that point, a sound input unit 414 that receives input of the sound signal output from the mic 32, a sound discrimination unit 415 that discriminates that a sound pressure having a predetermined level or more is detected in a frequency band that is considered to be a constituent element of snore by measuring the frequency of the sound signal input to the sound input unit 414, a decision unit 418 that decides whether or not snoring occurs based on discrimination results made by the sound discrimination unit 415 and information acquired by the waking information acquisition unit 411, and an output processing unit 419 for transmitting results decided by the decision unit 418 to the display unit 20 and/or the communication unit 50 in order to display the results. The results decided by the decision unit 418 can be displayed on another device by being transmitted to an external device via the communication unit 50.

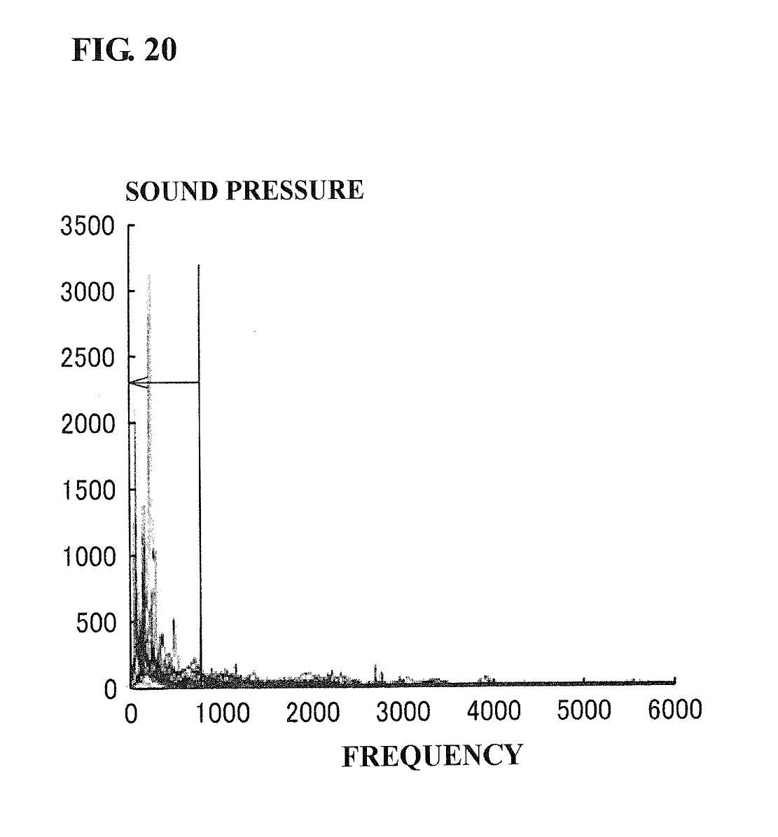

FIG. 20 is a diagram representing a result of analyzing a normal snoring sound.

Referring to FIG. 20, a normal snoring sound is constituted by a sound of approximately 1000 Hz or below. The decision unit 418 decides that snoring occurs based on a sound of 1000 Hz or below at a sound pressure having a predetermined level or more being detected in the sound discrimination unit 415 during a period in which the level acquired by the waking information acquisition unit 411 is in levels 1 to 3, for example.

Examples of Result Display

In the evaluation device 100, the CPU 41 discriminates a sleep level during a predetermined period. The CPU 41 then displays on the display unit 20 during the predetermined period the cumulative duration (sleeping state duration) from when it is discriminated that the sleep level is in any of levels 1 to 3 for the first time to the end of a period during which it is discriminated that the sleep level is in the sleeping state (sleep levels 1 to 3), and information indicating the occurrence of snoring in the case where the snoring occurs during the period. Here, the predetermined period may be a period stored in the memory 42 in advance (a period from the time when measurement starts to the time when the measurement ends, which will be described later), or a period determined by a person being measured operating the button group 10, or the like.

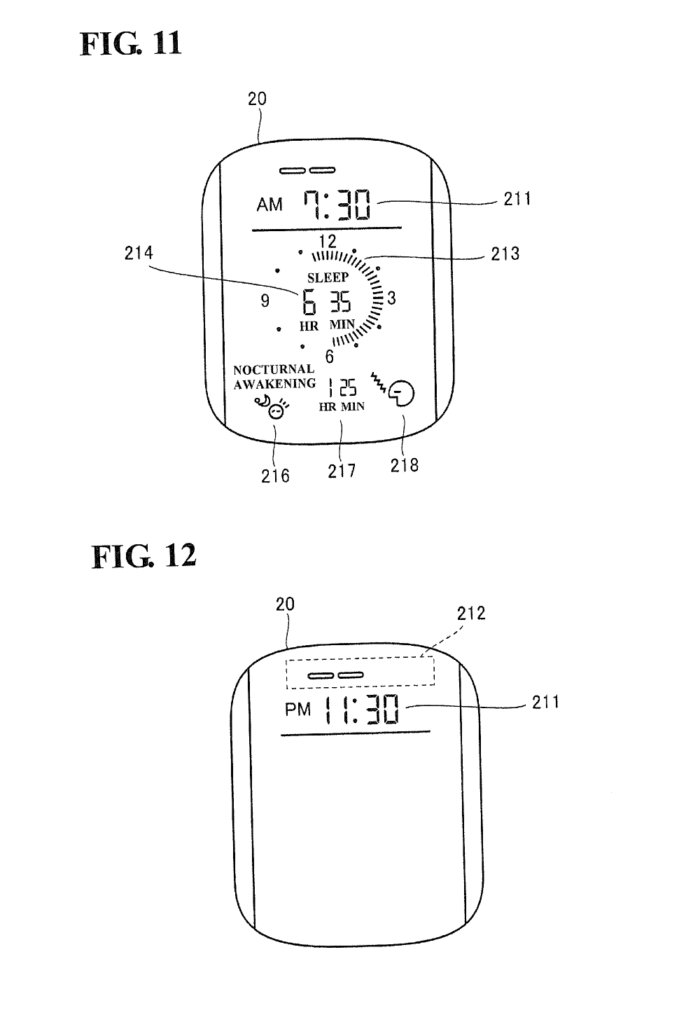

FIG. 11 is a diagram showing an example of the display modes of the display unit 20.

Referring to FIG. 11, the display unit 20 includes a display field 211 that displays current time, a display field 214 that displays sleeping state duration, and a display field 218 that displays an image showing the occurrence of snoring.

Note that in the above-described predetermined period, the period from a time point at which it is discriminated that the sleep level is any of levels 1 to 3 for the first time to when it is discriminated that the sleep level is an awakening state (described later) is referred to as a “sleeping duration” herein. The sleeping duration includes sleeping state durations and waking state durations. The above-described sleeping state durations are the cumulative durations of the durations in the sleeping state during the sleeping duration. In contrast, the cumulative durations of the durations in the waking state during the sleeping duration is referred to as “waking state duration”. Moreover, the display unit 20 includes a display field 213 for distinguishing and displaying the sleeping state and the waking state during the sleeping duration, a display field 216 that displays an image indicating the occurrence of the waking state during the sleeping duration, and a display field 217 that displays the number of hours (waking state duration) for which the waking state occurs during the sleeping duration.

Herein, the waking state occurring during the sleeping duration is referred to as “nocturnal awakening” as appropriate.

Examples of Display During Measurement

In the evaluation device 100, the CPU 41 displays on the display unit 20 that the detected signal output from the body motion sensor 31 is acquired for discriminating the sleep level during the above-described predetermined period.

FIG. 12 is a diagram showing an example of the display modes.

Referring to FIG. 12, the CPU 41 discriminates the level of the sleeping state for each unit period as described above, based on the detected signal output from the body motion sensor 31. In the example of display shown in FIG. 12, the CPU 41 discriminates the sleeping state level for each unit period about the detected signal output from the body motion sensor 31 immediately after acquiring the detected output signal. The display unit 20 also includes a display field 212 that displays the result of discriminating the sleeping state level. In the display field 212, a plurality of bars can be displayed. The CPU 41 discriminates the sleeping state level for each unit period and displays the number of bars corresponding to the discriminated level on the display field 212.

However, the CPU 41 may discriminate the sleeping state level for each unit period in accordance with the button group 10 being operated after acquiring the detected signal output from the body motion sensor 31, and it is not necessary to discriminate the sleeping state level immediately after acquiring the detected output signal. In such a case, it is not necessary to display the display field 212 in the display unit 20 during the sleeping duration.

Overall Operation

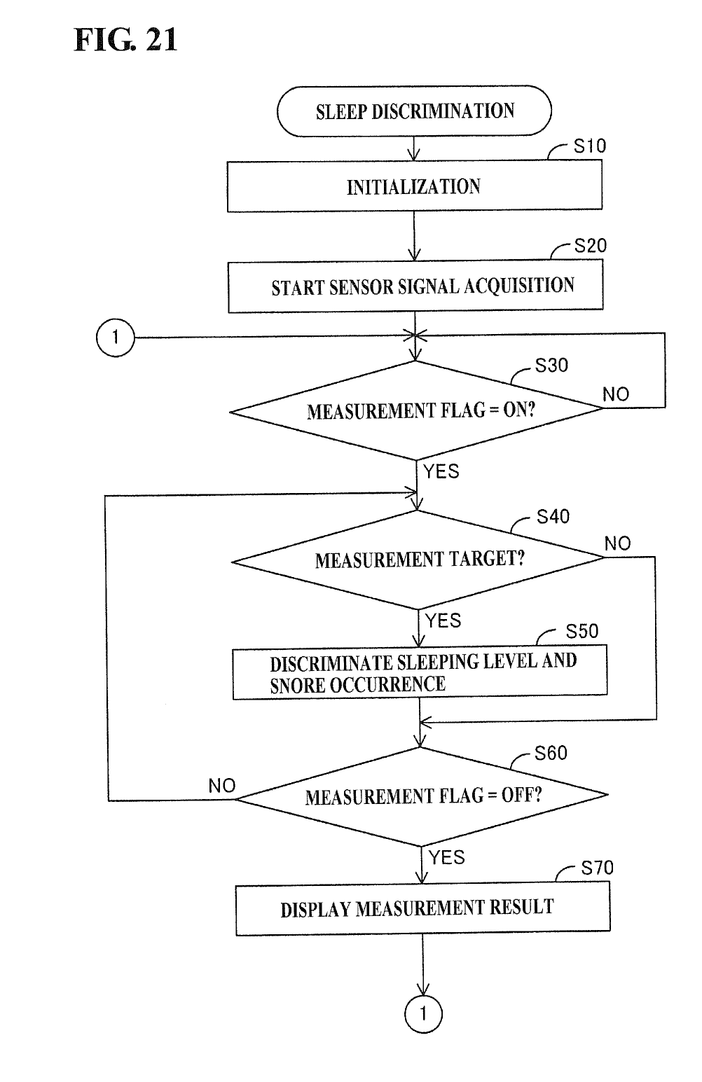

FIG. 21 is a flowchart of processing for discriminating a sleeping state (sleep discrimination processing) of a person being measured in the evaluation device 100. The processing is started when the evaluation device 100 is turned on, for example. Note that the operation of the processing is realized by the CPU 41 reading out and executing a program for display stored in the memory 42 so as to cause the functions shown in FIG. 6 to work.

In the sleeping discrimination processing, the CPU 41 sets a measurement flag to ON or OFF. In the initial state of the evaluation device 100, the state of the measurement flag is set to OFF. The function of the flag will be described later.

Referring to FIG. 21, when the evaluation device 100 is turned on, the CPU 41 initializes the evaluation device 100 in step S10, and advances the processing to step S20.

The CPU 41 starts acquisition of a sensor signal using the body motion sensor 31 in step S20, and advances the processing to step S30.

The CPU 41 determines whether or not the state of the measurement flag is ON in step S30, and advances the processing to step S40 if it is determined to be ON. On the other hand, if the state of the measurement flag is OFF, then the CPU 41 waits in step S30 until it is set to ON. Note that the CPU 41 changes the state of the measurement flag to ON as a result of the button group 10 (a specific button included in the button group 10) being operated, and, on the condition that the measurement start time that is stored in the memory 42 in advance has been reached.

In step S40, the CPU 41 discriminates whether or not a target to be measured is present in a range within which the body motion sensor 31 detects the target; in other words, whether or not the person being measured is present within range of the radio waves output from the body motion sensor 31. This discrimination can be realized based on whether or not the amplitude of either the respiratory waveform or the body motion waveform after the signal output from the motion sensor 31 is separated into the respiratory waveform and the body motion waveform continues to be smaller than a specific value for a specific time period (for example, 30 seconds). In the case where it is determined that the person being measured is present, in other words, in the case where it is discriminated that the state of the person's presence or absence is the state (E), the CPU 41 advances the processing to step S50. Note that in the case where it is determined that the person being measured is not present, in other words, in the case where it is discriminated that the state of the person's presence or absence is the state (N), the CPU 41 advances the processing to step S60 without performing the process of step S50.

The CPU 41 discriminates the sleep level and whether or not snoring occurs in step S50, and advances the processing to step S60. Note that in step S50, for example, the CPU 41 discriminates the sleeping state in each preset unit period, based on the periodicity of the respiratory waveform and/or the magnitude of the amplitude of the body motion waveform obtained in step S40 for each preset unit period. Furthermore, the discrimination results are corrected according to the discrimination results of the adjacent unit periods. Moreover, with respect to a fixed period consisting of the above-described successive unit periods, the sleep level is discriminated based on the discrimination result of each unit period. The obtained sleep level is then stored in the memory 42 along with time information corresponding to the sleep level.

The CPU 41 determines whether or not the state of the measurement flag is OFF in step S60, and advances the processing to step S70 if it is determined to be OFF. On the other hand, if it is determined to be ON, the CPU 41 returns the processing to step S40.

Note that the CPU 41 sets the state of the measurement flag to OFF as a result of the button group 10 (a specific button included in the button group 10) being operated when the state of the measurement flag is ON. Also, the CPU 41 may set the state of the measurement flag to OFF based on the detected signal output from the body motion sensor 31. For example, in the case where the state (N) continues for a predetermined time period in step S40, the CPU 41 may set the state of the measurement flag to OFF. The determination made by the CPU 41 that the state of the measurement flag is set to OFF corresponds to the discrimination made by the CPU 41 that the person being measured is in an awakening state.

The CPU 41, in step S70, causes the display unit 20 to display the result of discriminating the sleep level or the like in a period from when it is determined that the state of the measurement flag is ON in step S30 to when it is determined that the state of the measurement flag is OFF in step S60, and then returns the processing to step S40.

In the sleep discrimination processing described above, the sleep level is discriminated during the period in which the state of the measurement flag is set to ON. This period corresponds to the above-described predetermined period.

Note that in the above-described sleep discrimination processing, the measurement result is displayed on the display unit 20, in response to determining that the state of the measurement flag returns to OFF (at the time of determination of YES in step S60). The measurement result may not be necessarily displayed at this timing, or may be displayed at a timing at which the button group 10 is operated, or the like after the state of the measurement flag is set to OFF.

Also, the measurement result may be displayed on the display unit 20, or may be displayed on another device such as the display device 200 that has acquired the required data from the CPU 41 or the like.

Display Example of Display Unit in Sleep Discrimination Processing

An example of changes in display content of the display unit 20 is now described below, in the sleep discrimination processing described with reference to FIG. 21.

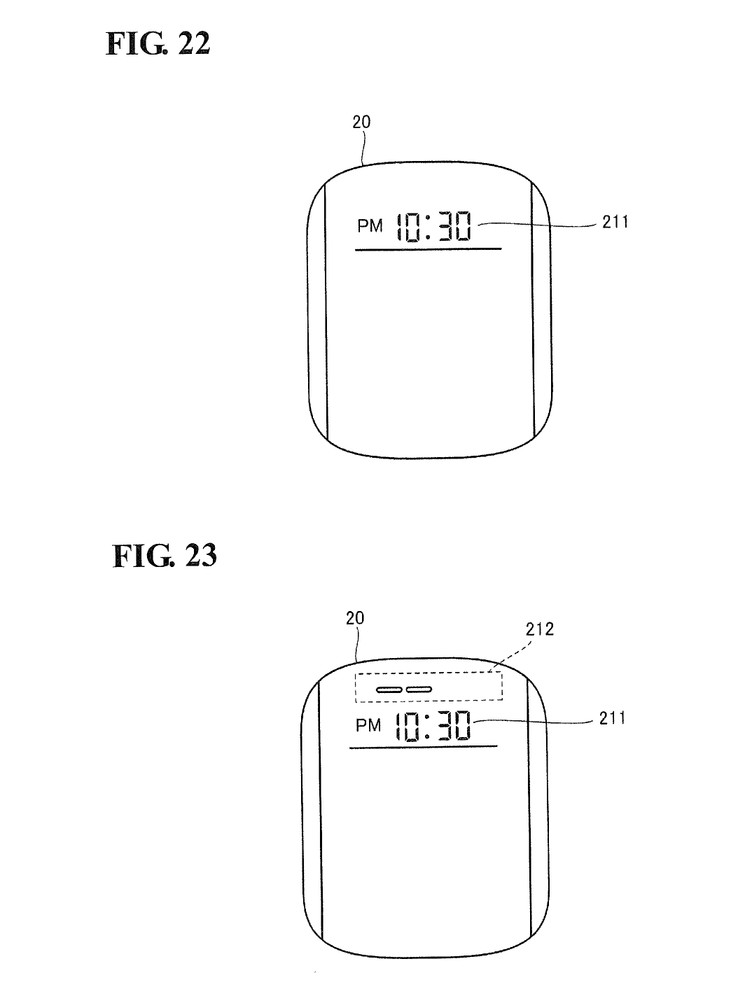

(FIG. 22: at the time of starting measurement)

If it is determined in step S30 that the state of the measurement flag is ON, the time at that time point is displayed in the display field 211 of the display unit 20, as shown in FIG. 22. Note that for a few seconds immediately after it is determined that the state of the measurement flag is ON, the CPU 41 may increase the brightness of the entire screen of the display unit 20 so as to notify that the discrimination of the sleep level is started.

(FIG. 23: during a measurement period)

Thereafter, if the processing is advanced to step S40, the time at this point is displayed in the display field 211 of the display unit 20 as shown in FIG. 23, and furthermore the result of discriminating the sleeping state level at the point is displayed in the display field 212.

Display shown in FIG. 23 continues until it is determined that the state of the measurement flag is OFF in step S60. Display in the display field 212 is updated as appropriate in accordance with changes in the results of discriminating the sleeping state level.

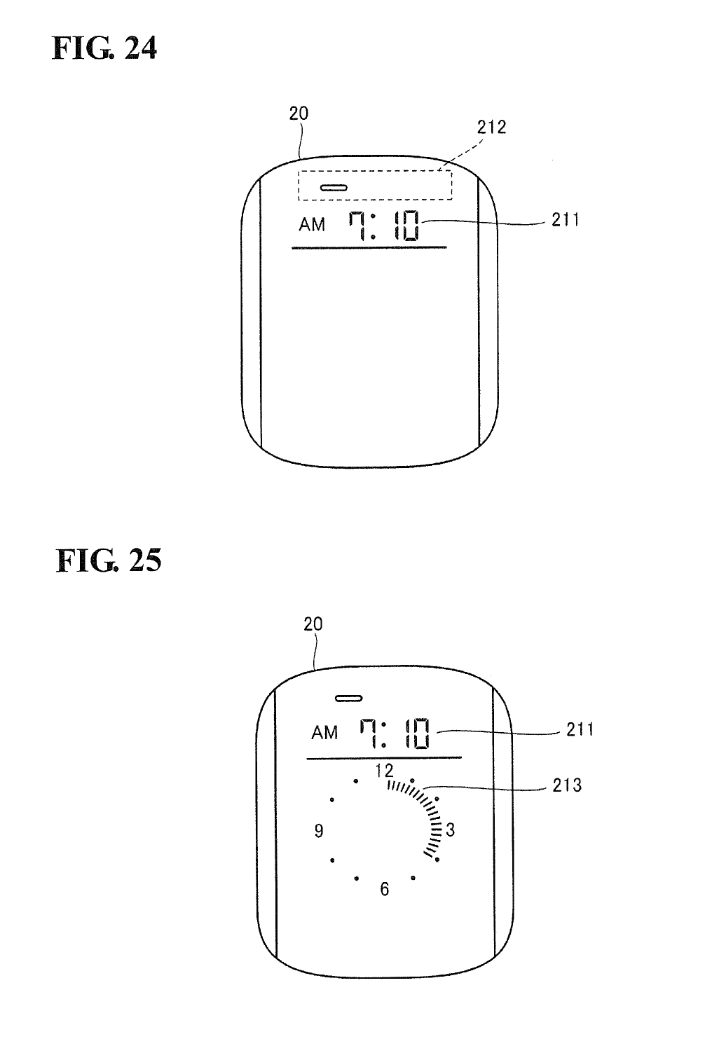

(FIG. 24: at the time of ending measurement (1))

If it is determined in step S60 that the state of the measurement flag is OFF, the CPU 41 displays the time at this point in the display field 211 of the display unit 20 as shown in FIG. 24, and temporarily (a few seconds, for example) increases the brightness of display of the entire display unit 20, in a state in which the result of discriminating the sleeping state level at this point is displayed in the display field 212.

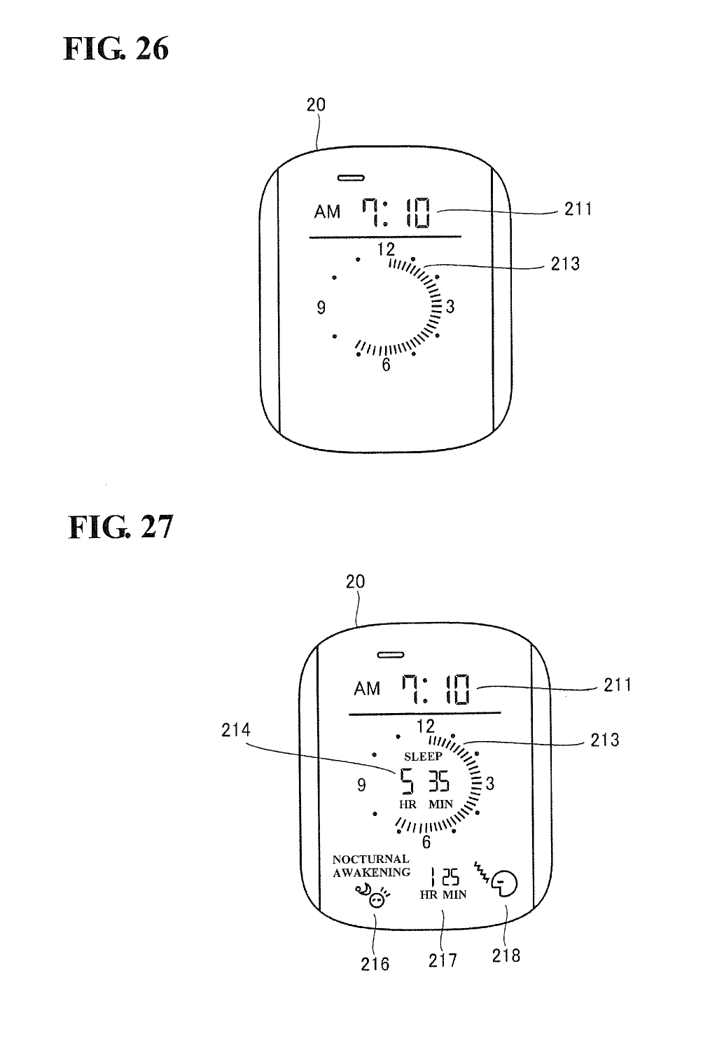

(FIGS. 25 and 26: display of sleeping duration)

Thereafter, the information representing the above-described sleeping duration is displayed in the display field 213.

The display field 213 is a field for displaying a plurality of bars arranged circularly. The plurality of bars, which are arranged in 360 degrees, are to be arranged so as to correspond to 12 hours. Five bars correspond to 1 hour. In other words, one bar corresponds to 12 minutes. Therefore, sixty two bars at the maximum can be displayed in the display field 213.

Displaying the information representing the sleeping duration in the display field 213 is realized by displaying the number of bars and the positions thereof that correspond to sleeping duration, among the plurality of bars arranged circularly (in 360 degrees) for example. In the present embodiment, a plurality of display units arranged circularly are composed by such bars.

FIG. 26 shows the display example of the display unit 20 for this case. The bar corresponding to 12:10 p.m. to the bar corresponding to 7:10 a.m. in the next morning are displayed in the display field 213 of FIG. 26.

Note that when the display shown in FIG. 26 is made, the display may be made through the display shown in FIG. 25, in other words, may be made such that bars to be displayed are displayed sequentially in a clockwise direction. It is possible to emphasize and display on the display unit 20 the length of sleeping duration to a person who looks at the display of the sleeping duration such as the person being measured or the like due to the bars being displayed in this manner. Therefore, the impression that the person being measured has with regard to the length of his or her sleeping time can be strengthened, and thus, his or her satisfaction with regard to sleep can be improved.

As described above, in the present embodiment, the sleeping durations are displayed through display or non-display of the plurality of bars, and the plurality of bars are arranged circularly so as to represent a clock.

(FIG. 27: display of sleeping state duration and the like)

When ending display of the sleeping duration described above, the CPU 41 further causes the display unit 20 to display the above-described sleeping state duration, waking state duration, and information indicating the occurrence of snoring in the case where the snoring occurs during the sleeping duration. FIG. 27 is a diagram showing an example of display content of the display unit 20 at this time.

Referring to FIG. 27, the sleeping state duration is displayed in the display field 214, the waking state duration is displayed in the display field 217, and an image indicating the occurrence of snoring is displayed in the display field 218. Note that FIG. 27 displays the measurement results of the case where the state of the measurement flag was set to ON at 10:30 p.m., it was discriminated that the sleep level of the person being measured was a level corresponding to the sleeping state (levels 1 to 3) at 12:10 a.m. in the next morning for the first time, the state of the measurement flag was set to OFF at 7:10 a.m., and then the waking state duration in the period from 12:10 a.m. to 7:10 a.m. were 1 hour and 25 minutes. In this case, because the sleeping duration is 7 hours, the sleeping state duration is 5 hours and 35 minutes, which is obtained by subtracting the waking state duration (1 hour and 25 minutes) from the sleeping duration (7 hours).

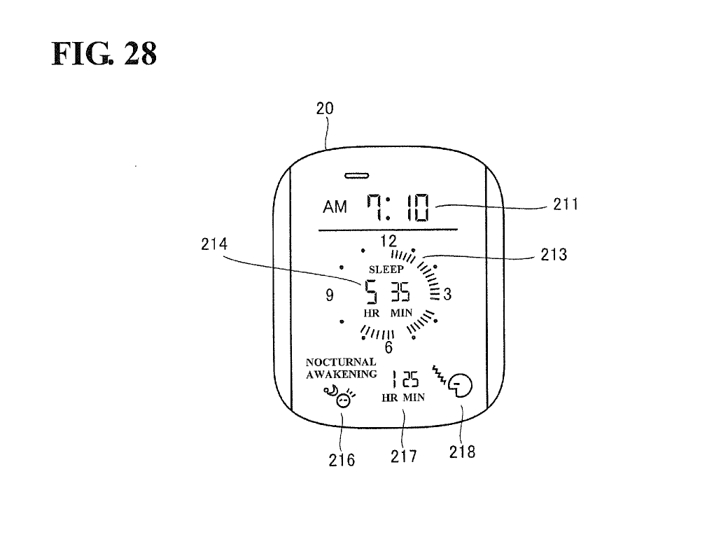

(FIG. 28: blinking display of waking state duration)

After displaying the sleeping state duration and the like on the display unit 20 as shown in FIG. 27, the CPU 41 may further display in a blinking manner the bars corresponding to the waking state duration among the bars corresponding to the sleeping duration displayed in the display field 213. In FIG. 28, a state is shown in which the bars corresponding to the waking state duration among the bars corresponding to the sleeping duration displayed in FIG. 27 are not displayed temporarily due to blinking.

As described above, in the present embodiment, after the sleeping duration shown in FIG. 27 are displayed in the display field 213, the bars corresponding to the waking state duration in the sleeping duration are displayed in a blinking manner by alternating between the display of FIGS. 27 and 28 repeatedly. This blinking display may be stopped by an operation from the outside such as an operation on the button group 10 or the like, or may be stopped after continuing for a fixed time period.

The embodiments disclosed herein are to be considered in all respects as illustrative and not restrictive. The scope of the invention is not defined by the above description but by the claims, and all changes that come within the meaning and range of equivalency of the claims are intended to be embraced therein.

### REFERENCE SIGNS LIST

10 button group

20 display unit

30 sensor

31 body motion sensor

32 mic

40 control unit

41 CPU

42 memory

50 communication unit

100 evaluation device

200 display device

401 input unit

402 first discrimination unit

403 decision unit

404 generation unit

405 storage unit

406 readout unit

407 display control unit

408 communication control unit

409 second discrimination unit

410 input information processing unit

411 waking information acquisition unit

414 sound input unit

415 sound discrimination unit

4021 discrimination unit

4022 correction unit

1. A sleep evaluation device comprising:
a body motion detection device for detecting body motion of a person being measured on a bed;
a first discrimination device for discriminating a sleeping state of the person being measured in a first period, based on a detection result of the body motion detection device;
a second discrimination device for discriminating whether the person being measured is in the sleeping state or a waking state for each second period that is longer than the first period, based on a discrimination result of the first discrimination device; and
a display device for displaying a discrimination result of the second discrimination device on the display device,
wherein the display device further displays information relating to the occurrence of nocturnal awakening in the sleeping state on the display device, based on the discrimination result of the second discrimination device, and
the display device displays a plurality of display portions arranged circularly on the display device, and displays the discrimination result of the second discrimination device using the plurality of display portions, while associating each of the display portions to a fixed period.

2. The sleep evaluation device according to claim 1, wherein the information relating to the occurrence of nocturnal awakening is at least duration during which the nocturnal awakening occurs in the sleeping state or the number of times that the nocturnal awakening occurs in the sleeping state.

3. (canceled)

4. The sleep evaluation device according to claim 1, wherein the display device displays the display portions corresponding to a period that is discriminated to be either the sleeping state or the waking state and the display portions corresponding to a period that is discriminated to be the other state, in different display modes from each other.

5. The sleep evaluation device according to claim 4, wherein the display device displays in a lighting manner the display portions corresponding to the period that is discriminated to be either the sleeping state or the waking state, and displays in a blinking manner the display portions corresponding to the period that is discriminated to be the other state.

6. The sleep evaluation device according to claim 1,
wherein the first discrimination device discriminates the sleeping state of the person being measured for each first period, for a specific period that is longer than the first and second periods, and
the display device further displays on the display device cumulative duration of either the sleeping state duration or the waking state duration in the discrimination result of the second discrimination device in the specific period.

7. The sleep evaluation device according to claim 6,
wherein the second discrimination device further discriminates an awakening state of the person being measured, based on the discrimination result of the first discrimination device, and
the display device displays the information relating to the occurrence of nocturnal awakening in the sleeping state on the display device, in response to the second discrimination device discriminating the awakening state of the person being measured.

8. The sleep evaluation device according to claim 6, further comprising an input device that receives input of information designating the end of the specific period,
wherein the display device displays the information relating to the occurrence of nocturnal awakening in the sleeping state on the display device, in response to the input device receiving the input.

9. The sleep evaluation device according to claim 1, further comprising a detection device that detects the occurrence of snoring of the person being measured,
wherein the display device further displays on the display device information indicating that the occurrence of snoring is detected in a case where the detection device detects snoring of the person being measured.

10. The sleep evaluation device according to claim 1, wherein the display device further displays the discrimination result of the first discrimination device on the display device.

11. A sleep evaluation method that is to be executed in a sleep evaluation device including a body motion detection device for detecting body motion of a person being measured on a bed, the sleep evaluation method comprising:
a step of discriminating a sleeping state of the person being measured in a first period, based on a detection result of the body motion detection device;
a step of discriminating whether the person being measured is in the sleeping state or a waking state for each second period that is longer than the first period, based on the discrimination result in the first period; and
a step of displaying the discrimination result in the second period on a display device,
wherein the displaying step includes:
a step of displaying information relating to the occurrence of nocturnal awakening in the sleeping state on the display device, based on the discrimination result in the second period;
a step of displaying plurality of display portions arranged circularly on the display device; and
a step of displaying the discrimination result using the plurality of display portions, while associating each of the display portions to a fixed period.
